(19) United States
(12) Patent Application Publication
(10) Pub. No.: US 2026/0072930 A1
(43) Pub. Date: Mar. 12, 2026

(54) Computer System and Method for Providing a Subject-Related Data Development Platform

(71) Applicant: LizAI Inc., Newton, MA (US)

(72) Inventors: Trung Tin NGUYEN, Saarbrücken (DE); David R. ELMALEH, Newton, MA (US); Duy Khiet HO, Quincy, MA (US)

(73) Assignee: LizAI Inc., Newton, MA (US)

(21) Appl. No.: 18/828,300

(22) Filed: Sep. 9, 2024

(51) Int. Cl. G06F 16/25

(52) U.S. Cl. CPC G06F 16/254

(57) ABSTRACT

A method, performed by a computer system connected to a network, comprises processing at least one input data object for standardizing subject-related information. The method further comprises subjecting the subject-related information contained in the processed at least one input data object to a first machine learning model for generating a uniform dataset containing the subject-related information in a uniform structured format, and storing the uniform dataset in one or more secured data repositories connected to the network. The method further comprises providing a secured virtual environment accessible to users connected to the network, the secured virtual environment enabling importation of datasets stored in the one or more secured data repositories and a use of imported datasets as part of one or more user-controlled subject-related data development operations for generating at least one workspace-developed data object.

### BACKGROUND OF THE DISCLOSURE

In subject-related development fields, in particular the life sciences, such as biotechnology, medicine and healthcare, though not limited thereto, availability of systematic information, including factors and causalities, is vital, for example, for the development of targeted and/or individualized processes, such as medical treatment schemes, and/or agents, such as drugs. However, obtaining systematic information is often difficult in such fields due to the complexity of the processes involved and diverse individual conditions in real scenarios. Systematic knowledge is often achieved in such fields by combining large amounts of data, which, in turn, is often not available to either enterprise, corporation, or private users, including private individuals, commercial developers of subject-related processes and/or products, etc.

Furthermore, while a large amount of subject-related data mostly exists, for example, sample measurement records, individual reports and diagnostics, individual prescriptions, large-scale (demoscopic) surveys, public statistics, published research articles, etc., such data varies regarding the information type which it represents, including audio information, photographic information, handwritten notes, printed text, electronic character strings, etc., and regarding its accessibility, for example, maintained in an individual's private domain, published on the Internet, etc.

Furthermore, especially in a health-related context, but also in other subject-related fields, increased privacy regulations typically apply concerning a use of personal information. In addition, many individuals prefer not providing their personal subject-related data to third parties. This poses further burdens to subject-related data development.

### BRIEF SUMMARY OF THE DISCLOSURE

There is thus a need for a technique which facilitates improved subject-related data development.

Accordingly, there is provided a method according to claim 1, a computer program product according to claim 14, and a computer system according to claim 15.

According to an aspect, a method, performed by a computer system connected to a network, is provided. The method comprises receiving, by means of a data receiving module of the computer system and from at least one of a plurality of sources connected to the network, at least one input data object containing subject-related information according to at least one of a plurality of information types encoded in at least one of a plurality of data formats. The method further comprises processing, by means of a data extraction and classification module of the computer system, the at least one input data object for standardizing the subject-related information; subjecting, by means of a data engineering module of the computer system, the subject-related information contained in the processed at least one input data object to a first machine learning model for generating a uniform dataset containing the subject-related information in a uniform structured format; and, storing, by means of a storing module of the computer system, the uniform dataset in one or more secured data repositories connected to the network. The method further comprises providing, by means of a workspace module of the computer system, a secured virtual environment accessible to users connected to the network, the secured virtual environment enabling importation of datasets stored in the one or more secured data repositories and a use of imported datasets as part of one or more user-controlled subject-related data development operations for generating at least one workspace-developed data object.

Thus, the method permits provision of a data development platform, or workspace, which, by means of a correspondingly configured data extraction and classification module, is subject-related, and which is accessible for users, for example, by means of user communication devices, via a communication network, such as the Internet. As such, the method facilitates accessibility of the subject-related data development platform to a large group of potential users. An applicability of the method can thus be extended.

Furthermore, implementation of the method in connection with a networked environment facilitates receiving input data objects from a wide range of network-connectible sources for establishing the data development platform, for example, for training one or more machine learning models which may be used in performing one or more steps of the method. In addition, a processing of data objects containing subject-related information in any of multiple information types, such as acoustic information, image information and/or text information, can be facilitated by means of a correspondingly configured data extraction and classification module, thus further increasing a range and an amount of the subject-related information available. An effectiveness of the method, in particular a reliability and a suitability of data which is generated by means of the secured virtual environment, can be improved.

Furthermore, implementation of the method by means of a computer system facilitates an implementation, by an operator of the computer system, of (such as general) security rules, including privacy rules, regarding information contained in any data object which is received, stored and/or generated using the method and/or regarding an authorization of an individual user to access some or all of the functionalities of the method. This applies, in particular, in connection with data objects which are stored in the domain of an operator of the computer system.

Additionally, or alternatively, the method facilitates that any user connected to the computer system via the network is enabled to select individual security or privacy settings, for example, via a user terminal associated with a respective user, regarding subject-related data objects which are transmitted from the user, for example, by the user's own user terminal, to the computer system and/or which are generated using data objects relating to the user. In this way, a user is enabled to control a degree of privacy regarding information contained in data objects which may be transmitted to the computer system, thus increasing an acceptance of the method by a larger group of users and/or compliance of the method with legal security regulations.

Furthermore, the method facilitates that at least some functionalities of the method, in particular some or all of the processing, subjecting, storing and/or providing-steps, are performed in a domain of a user, for example, on a user terminal associated with a respective user. In this way, privacy of personal information contained in data objects associated with the user can be further improved, since personal or user-specific data may not need to be transmitted via the network. To this end, a provider of the method may provide corresponding computer software for installation on the user terminal(s). In such implementations, the user terminal(s) may be regarded as an extension of the computer system, which thus extends at least partially in the domain of the user(s).

Furthermore, by standardizing the subject-related information and generating, by means of a machine learning model, a uniform dataset containing the subject-related information in a uniform structured format, such as a tabular format organized by predefined subject-related information classes, the method facilitates a use of each uniform dataset, and of the information encoded therein, for any of various applications in connection with the one or more user-controlled subject-related data development operations for generating at least one workspace-developed data object. This may apply, in particular, independently from a specific information content of the received data object and of the information type. An application range, an effectiveness, and a usability of the method for subject-related data development can thus be further improved.

The plurality of information types may comprise at least one of image information, textual information, acoustic information, voice information, spreadsheet data, and/or database information, the database information including at least one of data generated by software and/or hardware, relational database information, object-oriented database information, and/or NoSQL database information.

The uniform dataset may be generated such that the uniform dataset complies with at least one privacy and/or security standard defined by legal regulations in one or more jurisdictions regarding the subject-related information.

Standardizing the subject-related information may comprise applying an error-detection-and-correction routine to the subject-related information.

The plurality of sources may comprise a plurality of source types.

The uniform structured format may comprise a uniform category-mapped format, in particular a uniform category-mapped tabular, diagram, chart and/or figure format.

The data development operations may include subjecting the imported datasets to at least one second machine learning model comprised by the workspace module.

The subject-related information may be life sciences-related, in particular health-related, information. Additionally, or alternatively, the subject-related data development operations may be life sciences-related, in particular health-related, data development operations.

Additionally, or alternatively, the subject-related information may be information from multiple domains, in particular comprising life sciences-related, more particularly health-related, health maintenance organizations-related, pharmaceutical technologies-related, biology-related and/or biotechnologies-related information. Additionally, or alternatively, the subject-related data development operations may be for data from multiple domains and comprise life sciences-related, in particular health-related, health maintenance organizations-related, pharmaceutical technologies-related, biology-related and/or biotechnologies-related data development operations.

The user-controlled subject-related data development operations may comprise receiving, from a user via the network, a life sciences-related, in particular health-related, pharmaceutical technologies-related, biology-related, biotechnologies-related, query, and the at least one workspace-developed data object comprises a life sciences-related, in particular health-related, pharmaceutical technologies-related, biology-related, biotechnologies-related, response to the life sciences-related query, the life sciences-related response for output by the computer system.

The life sciences-related query may be a health-related query, and the life sciences-related response may be a health-related response. Additionally, the health-related query and the health-related response may relate to at least one of a clinical condition and information of a person, medical information, a description of a medication, an interaction of medications, a mechanism of action of a medication, an underlying cause of a disease, a prediction of a disease development, a prevention of a disease development, medical treatment, personalized treatment, best fit treatment, a biological target for a treatment, and/or drug development.

The plurality of information types may comprise at least one of a handwritten note by a medical professional, a medical image, an electronic healthcare record, medical spreadsheet data, and/or medical database information, the medical database information including at least one of clinical data generated by software and/or hardware, relational database information, object-oriented database information, and/or NoSQL database information.

The one or more secured data repositories may comprise one or more secured data repositories hosted by an operator of the computer system and/or one or more secured data repositories hosted at one or more secured user domains of one or more users of the computer system.

According to another aspect, a computer program product is provided. The computer program product contains portions of program code which, when executed by a processor of a computer system, configure the computer system to perform the method as provided herein.

According to another aspect, a computer system is provided. The computer system comprises a processor and a data storage device operatively coupled to the processor, the data storage device containing portions of program code which, when executed by the processor, configure the processor to perform the following steps: receiving, by means of a data receiving module of the computer system and from at least one of a plurality of sources connected to a network, at least one input data object containing subject-related information according to at least one of a plurality of information types encoded in at least one of a plurality of data formats; processing, by means of a data extraction and classification module of the computer system, the at least one input data object for standardizing the subject-related information; subjecting, by means of a data engineering module of the computer system, the subject-related information contained in the processed at least one input data object to a first machine learning model for generating a uniform dataset containing the subject-related information in a uniform structured format; storing, by means of a storing module of the computer system, the uniform dataset in one or more secured data repositories connected to the network; and, providing, by means of a workspace module of the computer system, a secured virtual environment accessible to users connected to the network, the secured virtual environment enabling importation of datasets stored in the one or more secured data repositories and a use of imported datasets as part of one or more user-controlled subject-related data development operations for generating at least one workspace-developed data object.

### BRIEF DESCRIPTION OF THE DRAWINGS

Further details and advantages of the disclosure become apparent from the detailed description and the drawings. There is shown in:

FIG. 1 a computer system according to an example,

FIG. 2 a method according to an example, and

FIG. 3 processing an input data object by means of a data extraction and classification module according to an example.

### DETAILED DESCRIPTION OF THE DISCLOSURE

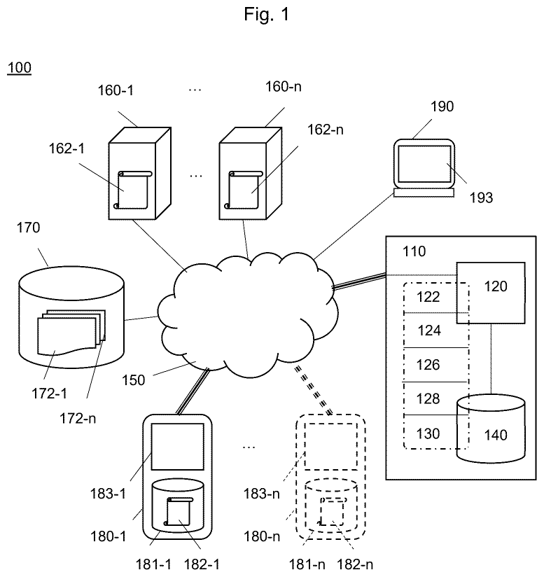

FIG. 1 shows schematically and exemplarily a networked computing environment 100. The networked computing environment 100 comprises a computer system 110, which is communicatively coupled to a network 150, such as the Internet.

The computer system 100 includes a processor 120 and a data storage device 140 operatively connected to the processor 120. The data storage device 140 stores program code which is executable by means of the processor 120. When the processor 120 executes the program code, the processor 120 performs operations which functionally constitute a data receiving module 122, a data extraction and classification module 124, a data engineering module 126, a storing module 128, and a workspace module 130, as will be described in more detail below.

Further connected to the network 150 is a plurality of network nodes, which include multiple servers 160-1, 160-2, 160-n. Each of the servers stores at least one data object 162-1, 162-2, 162-n, which pertain to one or more data repositories hosted by the servers 160-1, 160-2, 160-n. In some examples, at least some of the data objects 162-1, 162-2, 161-n pertain to a same data repository, such as a database which is managed by a third party and which is hosted in a distributed manner by various of the servers 160-1, 160-2, 160-n. Moreover, in some examples, each of at least some of the data objects 162-1, 162-2, 162-n belongs to a different data repository. The data objects 162-1, 162-2, 162-n are accessible via the network 150, for example, by means of a network browser of a computer system connected to the network 150, such as the computer system 110. In some examples, the data objects 162-1, 162-2, 162-n include one or more websites, which can be accessed and rendered by means of a network browser, such as public Internet websites, Internet websites with privileged access, etc.

The networked computing environment 100 further includes a user terminal 180-1 which is communicatively connected to the network 150. The user terminal 180-1 may include any type of user device which is suited for communicating data via the network 150, outputting received data towards a user via a user interface 183-1 of the user terminal 180-1 and receiving user input data from the user via the user interface 183-1 and transmitting received user input data via the network 150. Those types include mobile computing devices, such as smartphones, notebooks and/or tablet computers, stationary user terminals, such as computer stations with a user interface, etc.

The user terminal 180-1 comprises a data storage device 181-1, such as a built-in computer-readable non-volatile memory device. In the shown example, at least one subject-related data object 182-1 is stored on the data storage device 181-1. The user terminal 180-1 is configured to transmit the data object 182-1 towards components of the computer system 110 for use in a method as described herein. In some examples, the subject-related data object 182-1 contains encoded therein subject-related information which comprises personal and/or user-specific information of a user associated with the user terminal 180-1.

As indicated in FIG. 1 by the double lines, a data connection between the user terminal 180-1 and components of the computer system 110 via the network 150 is in some examples implemented as a secured data connection. In this way, security of transmitted user-related data and of data objects 182-2 stored by the user terminal 180-1 from unauthorized accessing is improved.

In an example, the subject-related information encoded in the comprises health-related information of a user of the user terminal 180-1, such as one or more of a medical record, diagnostic image data, a digitalized hand-written note by a doctor, a medical prescription, etc.

As indicated by the dashed lines in FIG. 1, the networked computing environment 100 may in some examples optionally include additional user terminals 180-n. Each of the additional user terminals 180-n is communicatively connected to the network 150 and comprises a data storage device 181-n with one or more subject-related data objects 182-n is stored thereon.

As indicated by the dashed lines in FIG. 1, the networked computing environment 100 may in some examples optionally include at least one subject-related, non-public database 170. The non-public database 170 stores non-public information which is encoded in one or more non-public data objects 172-1, 172-n. In an example, the non-public database 170 is a clinical database storing information about one or more patients, such as patient-specific bio-parametric data, patient-specific health data, patient-specific medical treatment data, non-patient-specific health data, non-patient-specific medical treatment data, etc., which are encoded in non-public data objects 172-1, 172-n.

The networked computing environment 100 further includes a developer terminal 190 which is communicatively connected to the network 150. The developer terminal provides in some examples the same functionalities as the user terminal 180-1 and may include any type of device which is suited for communicating data via the network 150, outputting received data towards a developer user via an interface 193 of the developer terminal 190 and receiving developer input data from the developer user via the interface 193 and transmitting received developer input data via the network 150.

In some examples, the developer terminal 190 is associated with a developer use who uses some or all of the functionalities provided by the computer system 110, as described below, without providing to the computer system 110 any subject-related data objects for processing in accordance with the techniques described herein. The developer user is, for example, a process and/or product developer, who uses the capacities of a virtual workspace provided by the computer system 110, including processed, in some examples anonymized, subject-related data which is stored by the computer system 110. The developer user is, for example, an authorized, such as registered, user of the functionalities offered by the computer system 170.

When in operation, the computer system 110 is configured to receive, by means of the data receiving module 122, input data objects containing subject-related information. The data objects include data objects such as the data objects 162-1-162-n stored by means of servers 160-1-160-n, data objects 172-1-172-n stored by means of nonpublic database 170, and/or data objects 182-1-182-n stored by means of user terminals 180-1-180-n. The subject related information comprises information relating to a subject for which the computer system has been configured to perform the techniques described herein, for example, life science-related information, health-related information, or the like.

In examples in which the subject-related information comprises health-related information, the data objects 162-1-162-n comprise one or more of published health statistics, published medical research, published medical surveys, and the like. Additionally, or alternatively, the data objects 172-1-172-n comprise nonpublic institutional medical data, such as clinical data. Additionally, or alternatively, the data objects 182-1-182-n comprise personal health-related data pertaining to a user associated with a respective user terminal 180-1-180-n, such as personal diagnostic data, digitalized handwritten notes by doctors, individual medical prescriptions, individual medical records, and the like.

In some examples, the data receiving module 122 is configured to receive data objects which have been transmitted from any of the network nodes, for example, upon input by a user of the corresponding network node, towards the data receiving module 122. Additionally, or alternatively, in some examples, the data receiving module 122 is configured to actively retrieve data objects, for example, by browsing data depositories connected to the network 150.

The computer system 110 is further configured to process each received input data object by means of the data extraction and classification module 124. The data extraction and classification module 124 is configured to process a range of coding formats and information types represented by the data objects, such as image information, handwritten notes, digital text, acoustic information, and the like, and to extract subject-related information encoded therein. To this end, the data extraction and classification module 124 employs in some examples optical character recognition as well as one or more machine learning models which have been trained for this purpose, etc.

In some examples, the data extraction and classification module 124 is further configured to identify subject-related entities in the processed data objects, such as, drugs, diseases, side effects, when used in the health-related context, and to generate metadata for each data object in which one or more of the identified entities is indicated.

In some examples, the data extraction and classification module 124 is further configured to classify the data object being processed based on the information types, identified entities, user characteristics, and the like, in accordance with any of multiple relevant categories of information for the subject-related data development platform, such as medical scans, doctor notes, or disease types, when used in a health-related context.

In some examples, the data extraction and classification module 124 is further configured to standardize the subject related information, including performing an error-detection-and-correction routine on the subject related information, and to organize and enrich the extracted and classified data for efficient queuing and analyzing, in particular to create and manage a structured representation of the processed information, for example, as part of a knowledge graph building process.

The computer system 110 is further configured to subject the subject-related information in the processed input data object to a machine learning model for generating a uniform data set which contains the subject-related information in a uniform structured format. To this end, the computer system 110 comprises a correspondingly configured data engineering module 126. Subjecting the information of the processed input data object to a generating of a uniform data set facilitates future use of the information, for example, in automatically creating a response to a user query.

In an example, the uniform structured format corresponds to a tabular format, in which the subject-related information is included, in the manner of an information template, in accordance with information classes. For example, when used in a health-related context, the information classes include different treatment parameters in a history of medical treatment of an individual user, or the like.

The computer system 110 is further configured to store, by means of the storing module 128, the uniform data set in a secure data repository 140, 181-1-181-n. In the case of personalized data, in some examples, the storing module 128 is configured to store the uniform data set in a secure storage device within a domain of the respective user, such as on data storage device 181-1 of the user terminal 180-1. In the case of anonymized data, which may be intended to be available to a process developer or a drug developer, such as developer user of the developer terminal 190, in some examples, the storing module 128 is configured to store the uniform data set in a secure storage device in a domain of an operator of the computer system 110, such as the data storage device 140.

The computer system 110 is further configured to provide, by means of the workspace module 130, a secure virtual environment accessible to users, such as users of the developer terminal 190 and/or users of any of the user terminals 180-1-180-n. The secure virtual environment, as exemplarily represented in FIG. 1 by the interfaces 183-1-183-n, 193, enables the importation of data sets stored in the secured data repositories 140, 181-1-181-n and the use of the imported data sets as part of user-controlled subject-related data development operations. Accessibility of a stored dataset to a particular user is determined, in some examples, depending on an authorization of the specific user in relation to a specific dataset and/or accessibility of the stored data set at its location of storage from the perspective of a given user device.

The user-controlled subject-related data development operations are configured for generating workspace developed data objects. For example, when used in a health-related context, the user-controlled subject-related data development operations include a personal user query which is input by a user of any of the user terminals 180-1-180-n, which results in a health-related response being generated and provided by means of the secure virtual environment. In another example, the user-controlled subject-related data development operations include requirements specifications being input by a drug developer, which results in product specifications generated by means of the secure virtual environment, and the like.

In some examples, the workspace module 130 includes at least one machine learning model for performing and/or supporting the data development operations executed on the imported data sets.

The above examples have been described in connection with FIG. 1, in which the computer system 110 is shown as a separate network entity. However, it will be understood that in other examples some or all of the above-described functionalities of the computer system 110 can be implemented on one or more of the user terminals 180-1-180-n and/or the developer terminal 190, respectively. For example, a provider of the method described herein provides software code to authorized users of the user terminals 180-1-180-n and/or of the developer terminal 190, wherein such software code is executable by means of the user terminals 180-1-180-n and/or the developer terminal 190 to configure the user terminals 180-1-180-n and/or the developer terminal 190 to perform some or all of the operations described herein.

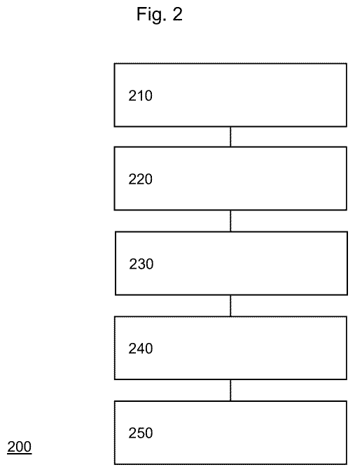

FIG. 2 shows a flow diagram of a method 200 performed by computer system connected to a network, such as computer system 110 in the networked computing environment 100 as described above in connection with FIG. 1.

The method 200 includes receiving, by the computer system and from at least one of a plurality of sources connected to the network, at least one input data object containing subject-related information according to at least one of a plurality of information types encoded in at least one of a plurality of data formats, step 210.

The method 200 further includes processing, by means of a data extraction and classification module of the computer system, the at least one input data object for standardizing the subject-related information, step 220. An example implementation of step 220 is described in more detail below in connection with the method 300.

The method 200 further includes subjecting, by means of a data engineering module of the computer system, the subject-related information contained in the processed at least one input data object to a first machine learning model for generating a uniform dataset containing the subject-related information in a uniform structured format, step 230, and, storing, by means of a storing module of the computer system, the uniform dataset in one or more secured data repositories connected to the network, step 240.

The method 200 further includes providing, by means of a workspace module of the computer system, a secured virtual environment accessible to users connected to the network, step 250. The secured virtual environment enables importation of datasets stored in the one or more secured data repositories and a use of imported datasets as part of one or more user-controlled subject-related data development operations for generating at least one workspace-developed data object.

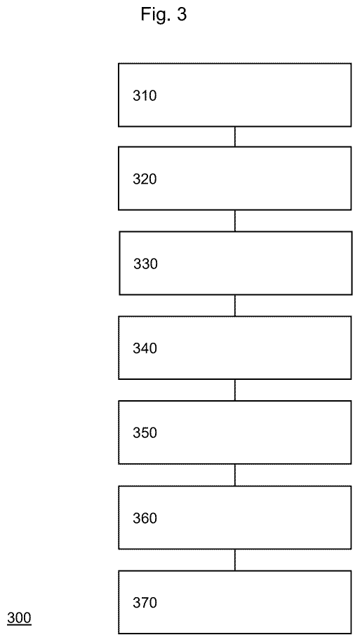

FIG. 3 shows a flow diagram of a method 300 for processing an input data object by means of a data extraction and classification module of a computer system. The method 300 provides an example implementation of step 220 of the method 200.

Upon receipt of the input data object by the data extraction and classification module, such as the data extraction and classification module 124, the method 300 comprises classifying a data format of the input data object, step 310. As a result, an information type of information contained in the input data object can be determined, such as image information, textual information, acoustic information, voice information, spreadsheet data, and/or database information.

The method 300 further comprises performing a structure analysis on the input data object, step 320. For example, when it has been determined in step 310 that the input data object comprises a PDF document, a layout analysis is performed on the PDF document. In another example, when it has been determined in step 310 that the input data object comprises database information, a database schema analysis for the database is performed.

The method 300 further comprises extracting data from one or more detected components of the input data object, step 330. For example, when the input data object comprises a PDF document, text and/or data is extracted from tables, figures, footers, editors, etc., which have been determined as a result of a layout analysis in step 320. In another example, when the input data object comprises database information, data is extracted from tables which have been determined as a result of a database schema analysis in step 320.

The method 300 further comprises detecting data extraction errors and correcting extracted data, using one or more machine learning models, step 340. For example, one or more machine learning models are used to correct errors which result from broken layouts, hard-to-extract handwriting, etc., and/or for improving data accuracy and context. In addition, various other types of errors are corrected in some examples, such as topographical mistakes, overlapping text, font problems, and the like.

The method 300 further comprises combining extracted data from one or more components, step 350. For example, an input data object can have multiple components from which data has been extracted in the preceding steps. Such data is combined in step 350.

The method 300 further comprises identifying domain specific entities and generating metadata, such as annotating or aliasing, for the extracted data, step 360. For this purpose, in some examples, a recognition of named entities and/or clinical variable identification is performed. This is facilitated, in some examples, by provision of a comprehensive list of entities and/or clinical information which can be identified. For example, when the method is used in a medical context, named entities such as drugs, diseases, side effects, relationships among the aforesaid entities, doses, and the like, can be recognized from handwritten doctor notes and, for example, can be converted into a structured format such as JSON.

The method 300 further comprises exporting the extracted data into an electronic file for subsequent operations, step 370. Subsequent operations comprise in some examples the generation of a uniform dataset by subjecting the electronic file to one or more machine learning models, in accordance with step 230 of the method 200.

In a first example scenario, the methods and systems described herein provide, for each user of a first user group, a secure and private virtual environment that allows a user to utilize the operator's built-in machine learning algorithms for visualizing and analyzing subject-related data, developing predictive models, or creating their own machine learning algorithms to work with subject-related uniform datasets. In such a scenario, the user-controlled subject-related data development operations for generating a workspace-developed data object in step 250 comprise, for example, generating a graphical data object which visualizes an analysis of the subject-related data, generating a predictive model or a machine learning algorithm, and the like.

In a second example scenario, the methods and systems described herein provide, for each user of a second user group, a trained machine learning-based assistant which is optimized for subject-related data searching, designed to be effectively securable by security and privacy technologies. It allows a user to efficiently locate and retrieve subject-related information from subject-related input data objects. Using at least one machine learning model for one or more of the processes described ensures that subject-related data remains secure and private while providing accurate insights and easy navigation through large volumes of data. In such a scenario, the user-controlled subject-related data development operations for generating a workspace-developed data object in step 250 comprise, for example, generating a graphical data object which visualizes the located and retrieved subject-related information, and the like. The second example scenario can be implemented simultaneously with the first example scenario, and the first user group and the second user group can overlap at least partially.

The above examples have been described mainly in a health-related context. However, it will be understood that some, or all, of the above-described functionalities and advantages are achievable also in connection with other subject-related fields of data development.

1. A method, performed by a computer system connected to a network, the method comprising:
receiving, by means of a data receiving module of the computer system and from at least one of a plurality of sources connected to the network, at least one input data object containing subject-related information according to at least one of a plurality of information types encoded in at least one of a plurality of data formats;
processing, by means of a data extraction and classification module of the computer system, the at least one input data object for standardizing the subject-related information;
subjecting, by means of a data engineering module of the computer system, the subject-related information contained in the processed at least one input data object to a first machine learning model for generating a uniform dataset containing the subject-related information in a uniform structured format;
storing, by means of a storing module of the computer system, the uniform dataset in one or more secured data repositories connected to the network; and
providing, by means of a workspace module of the computer system, a secured virtual environment accessible to users connected to the network, the secured virtual environment enabling importation of datasets stored in the one or more secured data repositories and a use of imported datasets as part of one or more user-controlled subject-related data development operations for generating at least one workspace-developed data object.

2. The method according to claim 1, wherein the plurality of information types comprises at least one of image information, textual information, acoustic information, voice information, spreadsheet data, and/or database information, the database information including at least one of data generated by software and/or hardware, relational database information, object-oriented database information, and/or NoSQL database information.

3. The method according to claim 1, wherein the uniform dataset is generated such that the uniform dataset complies with at least one privacy and/or security standard defined by legal regulations in one or more jurisdictions regarding the subject-related information.

4. The method according to claim 1, wherein standardizing the subject-related information comprises applying an error-detection-and-correction routine to the subject-related information.

5. The method according to claim 1, wherein the plurality of sources comprises a plurality of source types.

6. The method according to claim 1, wherein the uniform structured format comprises a uniform category-mapped format, in particular a uniform category-mapped tabular, diagram, chart and/or figure format.

7. The method according to claim 1, wherein the data development operations include subjecting the imported datasets to at least one second machine learning model comprised by the workspace module.

8. The method according to claim 1, wherein:
the subject-related information is life sciences-related, in particular health-related, information, and
the subject-related data development operations are life sciences-related, in particular health-related, data development operations.

9. The method according to claim 1, wherein:
the subject-related information is information from multiple domains, in particular comprising life sciences-related, more particularly health-related, health maintenance organizations-related, pharmaceutical technologies-related, biology-related and/or biotechnologies-related information, and
the subject-related data development operations are for data from multiple domains and comprise life sciences-related, in particular health-related, health maintenance organizations-related, pharmaceutical technologies-related, biology-related and/or biotechnologies-related data development operations.

10. The method according to claim 1, wherein the user-controlled subject-related data development operations comprise receiving, from a user via the network, a life sciences-related, in particular health-related, pharmaceutical technologies-related, biology-related, biotechnologies-related, query, and the at least one workspace-developed data object comprises a life sciences-related, in particular health-related, pharmaceutical technologies-related, biology-related, biotechnologies-related, response to the life sciences-related query, the life sciences-related response for output by the computer system.

11. The method of claim 10, wherein the life sciences-related query is a health-related query and the life sciences-related response is a health-related response, the health-related query and the health-related response relating to at least one of a clinical condition and information of a person, medical information, a description of a medication, an interaction of medications, a mechanism of action of a medication, an underlying cause of a disease, a prediction of a disease development, a prevention of a disease development, medical treatment, personalized treatment, best fit treatment, a biological target for a treatment, and/or drug development.

12. The method according to claim 8, wherein the plurality of information types comprises at least one of a handwritten note by a medical professional, a medical image, an electronic healthcare record, medical spreadsheet data, and/or medical database information, the medical database information including at least one of clinical data generated by software and/or hardware, relational database information, object-oriented database information, and/or NoSQL database information.

13. The method according to claim 1, wherein the one or more secured data repositories comprise one or more secured data repositories hosted by an operator of the computer system and/or one or more secured data repositories hosted at one or more secured user domains of one or more users of the computer system.

14. A computer program product containing portions of program code which, when executed by a processor of a computer system, configure the computer system to perform the method of claim 1.

15. A computer system comprising a processor and a data storage device (operatively coupled to the processor, the data storage device containing portions of program code which, when executed by the processor, configure the processor to perform the following steps:
receiving, by means of a data receiving module of the computer system and from at least one of a plurality of sources connected to a network, at least one input data object containing subject-related information according to at least one of a plurality of information types encoded in at least one of a plurality of data formats;
processing, by means of a data extraction and classification module of the computer system, the at least one input data object for standardizing the subject-related information;
subjecting, by means of a data engineering module of the computer system, the subject-related information contained in the processed at least one input data object to a first machine learning model for generating a uniform dataset containing the subject-related information in a uniform structured format;
storing, by means of a storing module of the computer system, the uniform dataset in one or more secured data repositories connected to the network; and
providing, by means of a workspace module of the computer system, a secured virtual environment accessible to users connected to the network, the secured virtual environment enabling importation of datasets stored in the one or more secured data repositories and a use of imported datasets as part of one or more user-controlled subject-related data development operations for generating at least one workspace-developed data object.
